(19) United States
(12) Patent Application Publication
(10) Pub. No.: US 2022/0370835 A1
(43) Pub. Date: Nov. 24, 2022

(54) COUGH AND SNEEZE FREEZE DEVICE

(71) Applicant: Homer Elias Calhoun, Bloomington, IL (US)

(72) Inventor: Homer Elias Calhoun, Bloomington, IL (US)

(21) Appl. No.: 17/328,345

(22) Filed: May 24, 2021

(51) Int. Cl. A62B 23/00; A62B 25/00

(52) U.S. Cl. CPC A62B 23/00; A62B 25/00

(57) ABSTRACT

An improved device for use in decreasing the transmission of viral and pathogenic materials by capturing the bodily fluids after a wearer sneezes. The device includes a housing having a bottom portion, a cover, and a base. The bottom portion having receiving members 28 for housing a filter and ventilation openings 32. The cover is removably secured to the first mid-section perimeter of the bottom portion and includes a pair of moveable doors that extend downwards when a nose guide 34 is engaged to receive the sneeze of a user.

### FIELD OF THE INVENTION

An improved device for use in decreasing the transmission of viral and pathogenic materials from a wearer is disclosed. In its preferred embodiment, the device is configured to catch the bodily fluid of a wearer that is released when the wearer sneezes.

### BACKGROUND OF THE INVENTION

In most cases, it has become an imperative to carry or wear devices to prevent the spread of viruses in schools, work environments, places of fellowship, healthcare settings, family gathers, public transportation, restaurants, and at locations where people congregate. In general, wearing a device of the type provided herein can help stop the spreading of viruses or from unknowingly spreading it to others. The devices are provided herein can be used to control the spreading of viruses in conjunction with other public control mechanisms prescribed by government Organizations such as the Centers for Disease Control and Prevention.

A large number of hygienic devices have been used as health control devices to manage the spread of viruses that emanate from sneezing or coughing. For example, U.S. Pat. No. 8910312B1 provides for a sneeze catching apparatus including a sleeve having a first open end, a second open end, and a perimeter wall. An openly positioned frame with side edges is provided with a pad including anti-bacterial properties to capture and destroy bacteria exhaled by a person.

A nasal drip-absorbing device is disclosed in U.S. Pat. No. 4244057. The nasal drip-absorbing device is designed for use in cold weather and includes an absorbent pad with a removable connection member at its underside and cooperates with a matching attachment member that can be permanently secured to an article of clothing such as a glove or a coat sleeve.

U.S. patent application Ser. No. 14267535 provides for a cough-catching apparatus including a body for securing to a hand of an individual, the body having an outer surface, an area on the outer surface having a material thereon, and a disinfectant infused into the material. The body may be a glove body. The glove body may have an opening for inserting a hand into the glove body and a plurality of finger openings. The area may be a palm area, a top of hand area, or may include both a palm area and a top of hand area.

It is believed, however, that none of the devices provided above or those that have been manufactured to manage the transmission concerns of a pandemic describe the present invention as prescribed herein. Developing an improved device for use in decreasing the transmission of viral and pathogenic materials from a person who sneezes addresses an unmet need in the art. In addition, the device is non-hampering, easily placed on a wearer's arm, pleasant to wear, and further prevents the release and further inhalation of viral or pathogenic materials that are released from sneezing.

To this end, the novel and distinctive features of the invention consist of an improved device for catching the bodily fluid of a wearer that is released when the wearer sneezes. The device is further characterized with the following novel features: (1) A cover being removably secured to the first mid-section perimeter of the bottom portion of a housing and having a nose guide and a cavity for attachment to a pair of moveable doors. The pair of moveable doors extend in a downward direction when the nose guide is engaged. (2) A pair of moveable doors having filter material being attached to the bottom surfaces thereof and for capturing and facilitating the concentration of the body fluid in the filter and below the cover of the device. (3) A filter having a circular open top and being constructed of filter material and including a top frame that is removably secured around the bottom interior portion of the housing and being held in place with securing assemblies thereon.

The invention will be described with reference to an improved device for catching the bodily fluid of a wearer that is released when the wearer sneezes. Among other features, the housing, cover, and filter can be designed to correlate with the plurality of devices that have been developed to address a pandemic across a broad spectrum of interstate commerce.

### BRIEF SUMMARY OF THE INVENTION

It is an advantage of the present invention to provide an improved device for use in decreasing the transmission of viral and pathogenic materials from a wearer and which in its preferred embodiment is configured to catch the bodily fluid of a wearer that is released when the wearer sneezes.

It is an additional advantage of the present invention to provide an improved device with a bottom portion having a first mid-section perimeter, an external surface, an internal surface, and at least two ventilation openings provided on the external surface.

It is an additional advantage of the present invention to provide an improved device having a cover that is removably secured thereon and a cavity for the moveable placement of a pair of moveable doors that extend in a downward direction when a nose guide is engaged.

It is an additional advantage of the present invention to provide an improved device having moveable doors with filter material being attached to the bottom surfaces for capturing and facilitating the concentration of body fluids in a filter and below the cover of the device.

It is still an additional advantage of the present invention to provide an improved device having a filter that is concave in shape and that is placed within the bottom portion of a housing and wherein the filter is held in place with securing assemblies that are in a receiving relation therewith.

It is still an additional advantage of the present invention to provide an improved device for catching the bodily fluid of wearer that is released when the wearer sneezes and wherein the device is removably attached to a wearer's hand by means of rotatable elbows that are attached to a buckle system and a clasp.

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting. In case of conflict with terms used in the art, the present specification, including definitions, will control.

Additional advantages and features of the present invention will become more apparent when considered in light of the following specification and drawings.

### BRIEF DESCRIPTION OF THE SEVERAL VIEWS OF THE DRAWINGS

The present embodiments are illustrated by way of the figures of the accompanying drawings, which may not necessarily be to scale, in which like references indicate similar elements, and in which:

FIGS. 1, 2 and 3 illustrate an exemplary view of the device with the housing embodiments, first and second elbow, buckle system, and clasp member.

FIGS. 4, 5, and 6 illustrate perspective views of the device with the pair of moveable doors in a downward position and the filter being visible in various positions.

FIG. 7 illustrates a cutaway view of the device showing the pair of movable doors in a horizontal position and in cover-relation to the filter.

FIG. 8 illustrates a cutaway view of the device showing the pair of movable doors in an enabled vertical position and the filter in a receiving position for body fluids.

### DETAILED DESCRIPTION OF THE INVENTION

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

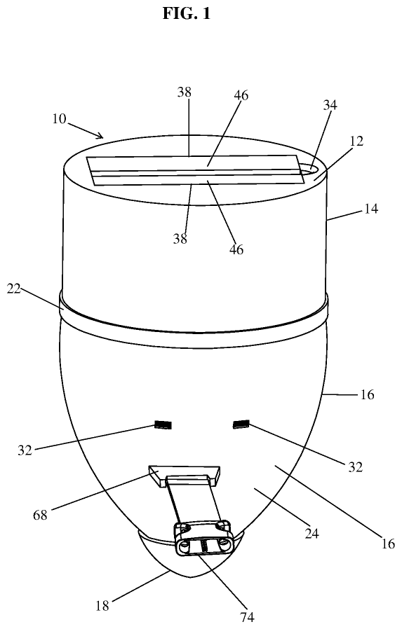

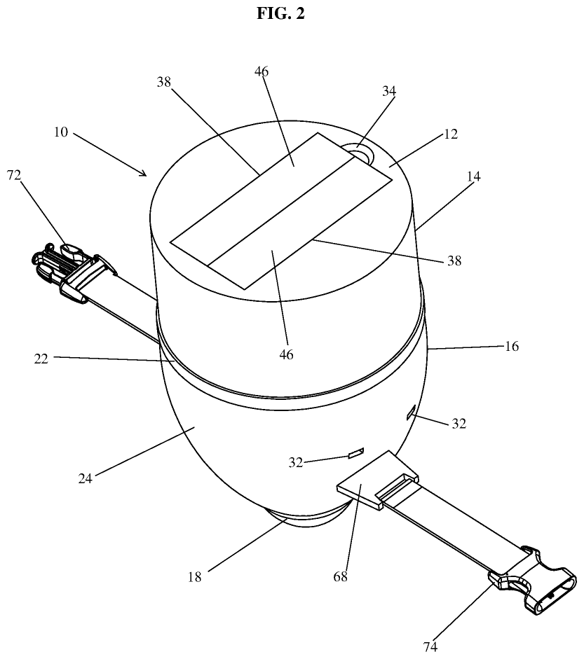

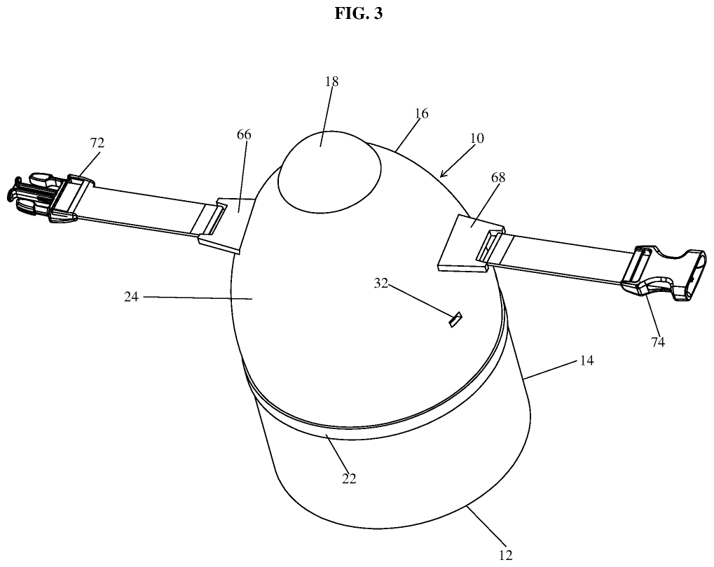

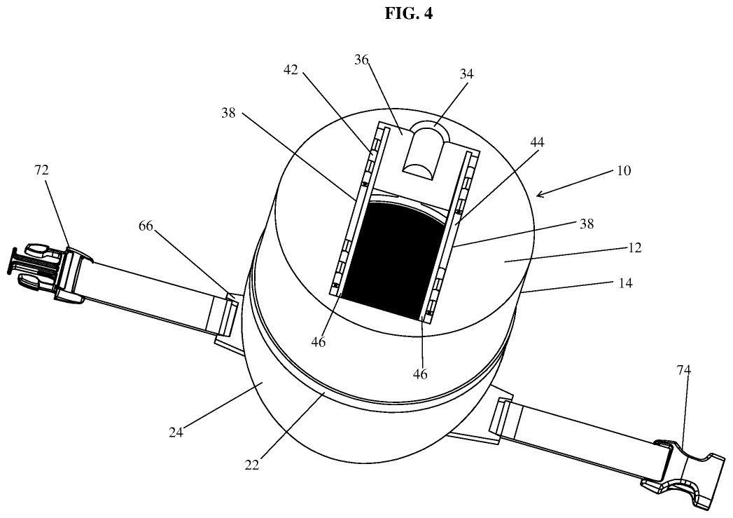

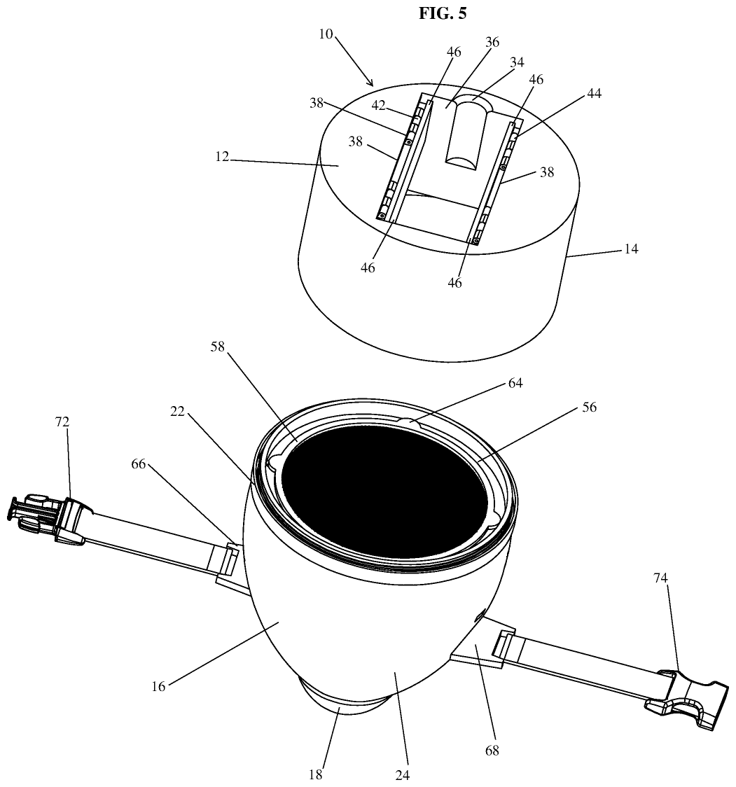

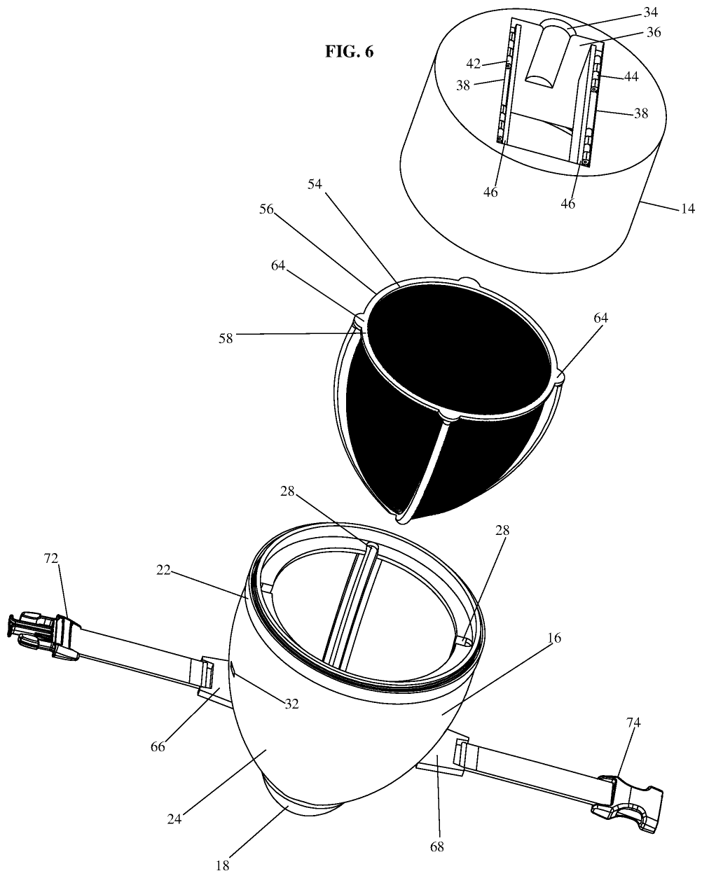

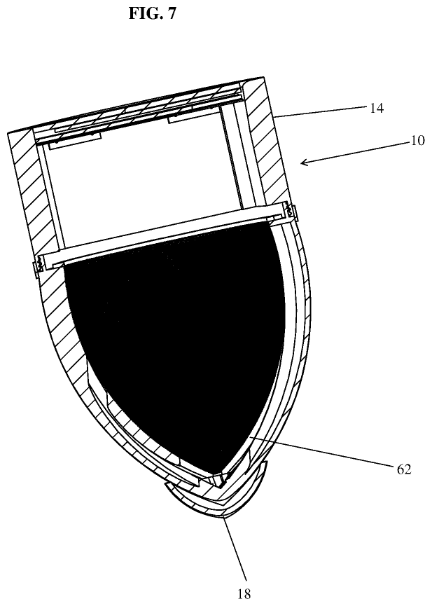

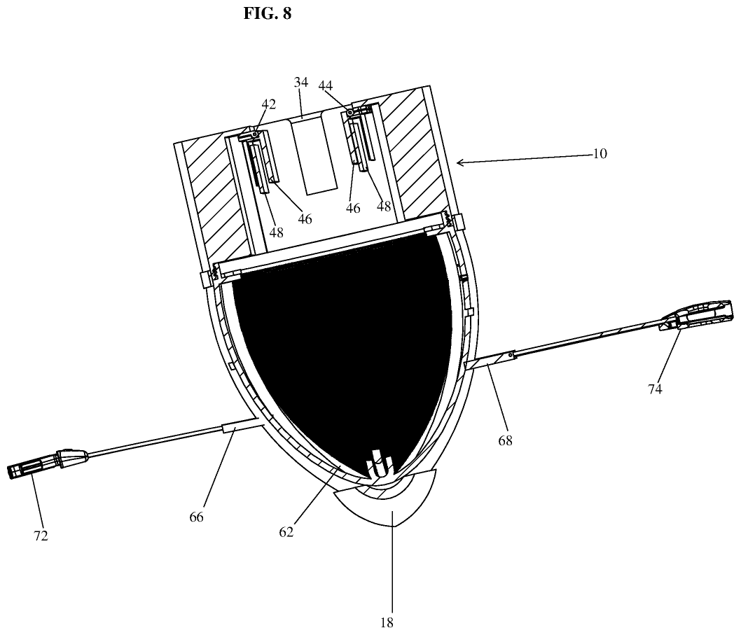

An improved device 10 for use in decreasing the transmission of viral and pathogenic materials from a wearer is disclosure. FIGS. 1-9 illustrate exemplary embodiments of the device 10 which in its preferred embodiment is configured to catch the bodily fluids of a wearer that is released when the wearer sneezes.

While a sneeze should be generally harmless, the bodily fluids that are released from the sneeze of an unhealthy person can spread thousands of droplets up to 6 feet away from the person. The sneeze droplets remain on surfaces and in the environment of the sneeze for 2 to 8 hours. These droplets typically range from 0.5 to 5 μm. It is well known that a single sneeze expels air with mucus containing irritants from the mouth and nose in an explosive and involuntary action. If the person is unhealthy, a virus then reenters the respiratory system of another individual when the droplets reach the eyes, nose or mouth, or when the hand touches the individual's face.

The device 10 that as provided herein addresses these transmission concerns and seeks to limit the negative influences of a sneeze by leaving a generally cleaner environment after a sneeze and cleaner surrounding air.

The housing 12 features a bottom portion 16, a cover 14, and a base 18. The bottom portion 16 of the housing 12 includes a first mid-section perimeter 22, an external surface 24, an internal surface 26, and at least two ventilation openings 32 provided on the external surface 24. The ventilation openings 32 are positioned at different locations thereon such that the ventilation openings 32 have different orientations to each other.

The cover 14 is removably secured to the first mid-section perimeter 22 of the bottom portion 16 and has a nose guide 34 and a cavity 36 with peripheral edges 38 including a left side 42 and a right side hinge 44 extending outwardly therefrom and for moveable attachment to a pair of moveable pair doors 46. The pair of moveable doors 46 extends in a downward direction when the nose guide 34 is engaged. The bottom surfaces 52 of the moveable doors 46 are attached to filter material 48 for capturing and facilitating the concentration of the body fluid in the filter and below the cover 14 of the device 10.

The filter 54 is placed within the bottom portion 16 of the housing 12 and includes a circular open top 56 and is constructed of multi-layered filter material 48. The filter 54 further includes a top frame 56 that is removably secured around the bottom interior portion 62 of the housing 12 and is held in place with securing assemblies 64 thereon. The filter 54 is held downward therein and wherein the filter 54 is a receiving face for the body fluids. The filter 54 assumes a substantially concave shape and the receiving member being in receiving relation with the top frame 56 of the filter 54. The filter 54 and the filter material 48 provided on the back surfaces of the pair of movable doors 46 can be constructed of multiple layers of absorbent material and backing sheets can be secured to the layers at their underside.

The bottom portion 16 of the housing 12 features a circular ring shown as a mid-section perimeter 22 to mask threading that can be provided therein. This mid-section perimeter 22 can be constructed from durable materials such as chrome. The base 18 functions as an arm rest, and in a preferred construction, the base 18 can be provided as a soft cushion that used for secure placement on a wearer's arm or in areas near the elbow. The device 10 is removably attached to a wearer's hand by means of a first 66 and a second rotatable elbow 68 that is attached to a buckle system 72 and a clasp member 74 respectively. The buckle system and clasp member permit easy detachment of the device 10 from the user's arm and re-attachment of the device 10 with a new filter 54 whereby the user can conveniently remove the used filter 54 without reaching into the bottom portion 16 of the housing 12. The pair of moveable doors 46 is positioned vertically above the filter 54 in a manner to conveniently use the device 10 and prevent body fluids from escaping therefrom.

In a preferred embodiment, the device 10 is lifted toward the nose of the user to capture the sneeze and such that the nose guide 34 is engaged for proper use thereof. However, the range of body fluids according to this invention can consists of the human grouping including sneezing, sweating, spitting, belching, coughing, etc. After use, the filter 54 should be quickly removed, discarded, replaced with another filter 54. The device 10 can be cleaned in the event of unwanted bodily fluids on the exterior surfaces or bodily fluids that might accidently collect on the interior surfaces thereof.

It should be further understood that the device 10 that is shown and in the figures and throughout this specification are exemplary and non-limiting. More or fewer embodiments can be added to the system to obtain as much structural and functional support as prescribed by the wearer. For example, the bottom portion 16 of the housing 12 is of concave shape and securely houses the filter 54 by sliding the filter 54 in a vertical manner along the interior surface of the housing 12. In order for the device 10 to be operable as a substantially leak-proof device, the shape and relevant thickness of the device 10 can be adjusted to achieve the same. In addition, the shapes and sizes of the filter 54 can be adjusted to facilitate replacement after the collection of variable body fluids that is collected therein. The device 10 is therefore designed for safety and accuracy of use. It will be apparent that the device 10 shown in the drawings may be also modified within the scope of the claims as to the relative size and shape of the elements. For example the pair of moveable doors can be constructed from a flexible rubber material the arms may be constructed much larger or much smaller than is shown in the drawings. In addition, the pair of moveable doors 46 is preferably moving in a downward direction. However, the pair of moveable doors 46 may be constructed for rearward sliding in the cover 14. It is also contemplated to have a pair of moveable doors 46 that can be readily unhinged and removed entirely or as desired by a user. However, the inventor prefers that the device 10 and all of the desired elements for using the same be sufficiently sized as described herein.

The selection of the materials to construct the device 10 will be determined based on properties including material density, texture, cost effectiveness, safety, resistance to cracking, etc. The materials for the filters as provided herein can be provided in a customary manufacture including the use of fabrics and multi-layered materials that are well known. And as required by the Centers for Disease Control and Prevention, the inventor will provide the specifications for the selected materials in packages that are produced in interstate commerce.

The device 10 can also be constructed in a plurality of colors, designs, shapes, and patterns to dictate the individualized tastes and needs of the manufacturer or users. Seals that are beneficial in a specific work environment where texture is required for health or safety reasons can also be used. In addition, a plurality of additional components such as logos, trademarks, family reunion decorations, sports memorabilia, and reflective materials can be applied and the fabric can be intermixed between various decorative patterns, knitting, and shape designs of materials typically used to construct the same.

This invention will be mass-produced within the United States and can be promptly procurable for use on worldwide scale. Accordingly, an object of the present invention is to provide a device 10 that is cost-effective, dependable, easily constructed, and relatively inexpensive. The device 10 can be made available for schools, churches, not-for-profit institutions, and in least-developed countries where the invention as prescribed herein is not easily manufactured or made commercially available.

Having described the invention, however, many modifications thereto will become apparent to those skilled in the art to which it pertains without deviation from the spirit of the invention as defined by the scope of the appended claims. Although the invention has been explained in relation to its preferred embodiment, it is to be understood that many other possible modifications and variations can be made without departing from the spirit and scope of the invention.

What is claimed is:

1. An improved device for use in decreasing the transmission of viral and pathogenic materials from a wearer and which in its preferred embodiment is configured to catch the bodily fluid of a wearer that is released when the wearer sneezes, the device comprising,
a) a housing having a bottom portion, a cover, and a base,
b) the bottom portion having a first mid-section perimeter, an external surface and an internal surface with receiving members, the bottom portion having at least two ventilation openings therein, said ventilation openings being positioned at different locations on the external surface thereof such that the ventilation openings have different orientations to each other,
c) the cover being removably secured to the first mid-section perimeter of the bottom wall of the housing and having a nose guide and a cavity with peripheral edges including left side and right side hinges extending outwardly therefrom and for moveable attachment to a pair of moveable doors, the pair of moveable doors extending in a downward direction when the nose guide is engaged,
d) the moveable doors having filter material being attached to the bottom surfaces for capturing and facilitating the concentration of the body fluid in the filter and below the cover of the device,
e) a filter being placed within the bottom portion of the housing, the filter having a circular open top and being constructed of filter material and including a top frame that is removably secured around the bottom interior portion of the housing and being held in place with securing assemblies thereon, the filter being held downward therein and wherein the filter is a receiving face and the filter assuming a substantially concave shape, said receiving member being in receiving relation with the top frame of the filter,
f) the device being removably attached to a wearers hand by means of a first and a second rotatable elbow that is attached to a buckle system and a clasp member respectively,

2. The device of claim 1 wherein the use thereof is a body fluid capturing event when a wearer sneezes.

3. The housing of claim 1 wherein the bottom portion of the housing has a generally concave configuration corresponding to but larger in size than the filter.

4. The cover of claim 1 wherein the pair of moveable doors of the cover is constructed to fit within the cavity of the cover.

5. The cover of claim 1 wherein the pair of moveable doors of the cover and the filter being provided thereon close over the filter of the container to provide a tight closure therewith.

6. The cover of claim 1 wherein the cover is removable and covers the filter.

7. The cover of claim 1 wherein the pair of moveable doors of the cover are of flexible construction.

8. The cover of claim 1 wherein the pair of moveable doors are identical in shape, have a generally rectangular configuration, and are provided on the same perpendicular plane to the other.

9. The filter of claim 1 wherein the filter is replaceable by moving the filter upward and by removing the securing assemblies of the filter from the securing members provided in the bottom portion of the housing.

10. The device of claim 1 wherein said first and said second elbow are of a semi-rigid construction.
